(19) United States
(12) Patent Application Publication
(10) Pub. No.: US 2007/0213280 A1
(43) Pub. Date: Sep. 13, 2007

(54) Steroidal Saponins Compound, the Process for Producing the Same and the Use Thereof

(75) Inventors: Xinsheng Yao, Guangdong (CN); Lianda Li, Guangdong (CN); Naili Wang, Guangdong (CN); Rongli Zhang, Guangdong (CN); Haifeng Chen, Guangdong (CN); Ping Shen, Guangdong (CN); Gexia Qu, Guangdong (CN)

Correspondence Address: GLOBAL IP SERVICES, 2462 ROCK ST., APT. 6, MOUNTAIN VIEW, CA, 94043, US

(73) Assignee: GUILIN INNOVATIVE MEDICINES INVESTMENT LTD., CO., Guilin, Guangxi (CN)

(21) Appl. No.: 10/599,156

(22) PCT Filed: Mar. 22, 2005
(86) PCT No.: PCT/CN05/00364

§ 371 Date: Sep. 21, 2006

(30) Foreign Application Priority Data
Mar. 22, 2004 (CN) 200410026557.9

(51) Int. Cl. A61K 31/7048; C07H 15/24; C07J 17/00

(52) U.S. Cl. 514/26; 536/6.2

(57) ABSTRACT

The present invention relates to a steroidal saponins compound, and the preparation and application in curing cardiovascular diseases, such as miocardial infarction, etc. The chemical structure of the steroidal saponins compound is as (I) or (II): 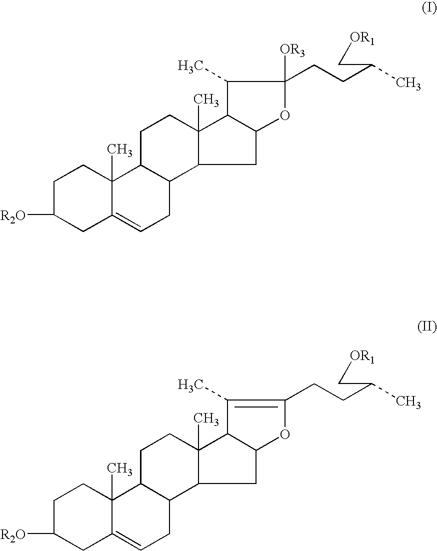
wherein, R₁=β-D-glucose; R₂=straight or bifurcate sugar chains; R₃═H or CH₃. In this invention, by various ways of separation, the steroidal saponins compound with chemical structure of (I) or (II) could be extracted, synthesized or semi-synthesized process from traditional Chinese medicine and natural products. Oral dosage drug or non-oral dosage drug was produced by the single steroidal saponins compound or the mixture of these compounds in vary ratio, the drug can be used to prevention and cure the diseases miocardial infarction, coronary artery disease, heart angina, arrhythmia, blood losing of cardiac muscle, hypertension, hyperlipaemia and ropy blood, etc. In structure (I) and (II), R₁=β-D-glucose; R₂=straight or bifurcate sugar chains; R₃═H or CH

### BACKGROUND OF THE INVENTION

1. Field of the Invention

This invention relates to a steroidal saponins compound, particularly, relates to Methylprotodioscin (MPD) and Pseudoprotodioscin (PPD), and the application of them in prevention and curing cardiovascular diseases, such as miocardial infarction (MI), etc.

2. Description of Prior Art

“Di'ao Xinxuekang” made by China Chengdu Di'ao Pharmacy Group has the efficacy of enhancing the dilatation of coronary artery blood vessel, and improving the effect of blood losing of cardiac muscle to rat. It is one of the best Chinese traditional medicines that prevention and curing cardiovascular diseases in the market of coronary artery disease treatment. The effective component of this medicine is steroidal saponins. The comparison on crude extracts of plant from this medicine to Discorea nipponica Makino or Discorea Panthaica Prainet Bukill by thinner liquid chromatography (TLC) was introduced in Chinese Pharmacopoeia (2000 Edition), and the components determination method was developed also to determine the weight of saponin in acid hydrolysis residue in order to find out sapogenin's total weight. Whether single furostanol saponin or mixture of two furostanol saponins in vary ratio will raise affection on alleviating miocardial infarction or not was neither reported nor disclosed in related patents or other publications.

### SUMMARY OF THE INVENTION

This invention relates to a new application of steroidal saponins compound on prevention and curing cardiovascular diseases, such as miocardial infarction, etc., the steroidal saponins compound has the chemical structures of (I) and (II) as below:

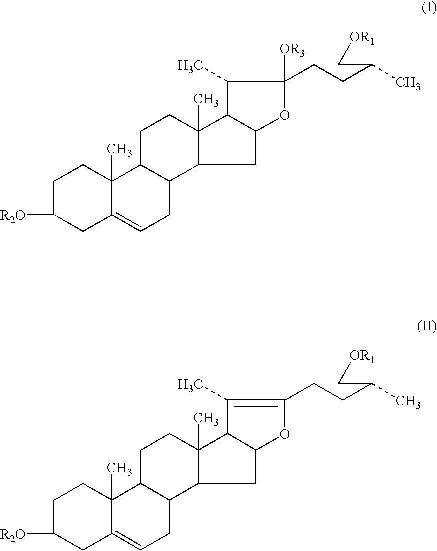

Wherein, R₁=β-D-glucose;

R₂=straight or bifurcate glycan chains, the glycan of the glycan chains involving β-D-glucose, α-D-glucose, α-L-rhamnose, β-D-galactose, α-D-galactose, β-D-mannose, α-D-mannose, β-D-arabinose, α-D-arabinose, β-D-xylose, α-D-xylose, β-D-ribose, α-D-ribose, β-D-lyxose, α-D-lyxose, α-D-fucose, and 6-deoxysugars and 2,6-dideozysugars corresponding to each of foresaid aldohexoses;

R₃═H or CH₃.

The steroidal saponin compound, Methylprotodioscin (MPD), with the structure of (I), has the application on prevention and curing cardiovascular diseases, such as miocardial infarction, etc., wherein the chemical structure of (I):

R₁=β-D-glucose;

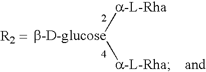

R₃═OCH3.

The steroidal saponin compound, Pseudoprotodioscin (PPD), with the structure of (II), has the application on prevention and curing cardiovascular diseases, such as miocardial infarction, etc., wherein the chemical structure of (II):

R₁=β-D-glucose

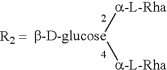

This invention applies the plant of Dioscorea genus as raw material, via various separation processes to isolate the purified and refined compounds, MPD and PPD. Single compound or the mixture of these two compounds was applied to experiment anti-miocardial-infarction effectivity on rat or dog, the results shown that MPD, PPD or the mixture of them in vary ratio could reduce the scope of miocardial infarction effectively, and has obvious effect on prevention and curing coronary artery disease. All about these have suggested the bright future of research and development of the steroidal saponin compound.

### BRIEF DESCRIPTION OF THE DRAWINGS

FIG. 1 shows the effect of MPD on miocardial infarction scope to rats;

FIG. 2 shows the effect of MPD on miocardial infarction scope to rats in repeat experiment;

FIG. 3 shows Effect of MPD on the miocardial infarction area of the canines;

FIG. 4 shows the comparison of the effect of MPD on canine's miocardial ischemia degree (N-ST) by each administration group (epicardial electrogram mensuration);

FIG. 5 shows the comparison of the effect of MPD on canine's miocardial ischemia degree (Σ-ST) by each administration group (epicardial electrogram mensuration);

FIG. 6 shows each administration group's influence on canine coronary arterial flow;

FIG. 7 shows each administration group's influence on canine miocardial consumption of oxygen (MCO);

FIG. 8 shows the percentage of MPD's influence on miocardial infarction area.

### DETAILED DESCRIPTION OF THE INVENTION

In the present invention, the plant of Dioscorea genus was used as raw material, Methylprotodioscin (MPD), Pseudoprotodioscin (PPD) and some other furostanol saponins with the foresaid structures of (I) or (II) were purified from the extract of the plant by various processes of separation, and synthesis of MPD was obtained in success also. By ways of thinner liquid chromatography (TLC) and high performance liquid chromatography (HPLC), furostanol saponins, including MPD and PPD, were confirmed as the components of “Di'ao Xinxuekang”. Considering the clinical apply of “Di'ao Xinxuekang”, the experiments on canine and rat's miocardial infarction were worked out by employing single furostanol saponin, such as MPD and PPD, or the mixture of them in various ratios, and the comparison of furostanol saponin with “Di'ao Xinxuekang” was made out also. The results showed that MPD, PPD or the mixture of them have obvious effect on improving canine miocardial infarction which caused by coronary artery ligation, and there's no significant statistical difference between “Di'ao Xinxuekang”.

### EXAMPLE 1

### Extraction and Isolation of Steroidal Saponin Compound MPD

Fresh rhizome of Discorea nipponica (70 kg) was extracted with 80% ethanol by heating refluxing; then concentrating the extract solution, and suspending the extract in water to get the dissolved portion and unsolved portion. Then the dissolved portion was passed through D101 absorbent resin column, and eluted by distilled water, 10%, 50% and 95% ethanol in order. The 50% ethanol eluted solution was concentrated, and be subjected to silica gel column chromatography (45˜75 um), then stepwise eluted by CH₃Cl/CH₃OH/H₂O solution (8:2.5:0.01) and methanol. The eluted solution be vaporized in vacuum and concentrated, and incorporate the crystals of component fractions of 46˜50, then re-crystal the crystalloid to get MPD compound (192.6 g).

### EXAMPLE 2

### Extraction and Isolation of Steroidal Saponin Compound PPD

Rhizome of Discorea futschauensis (3 kg) was extracted with 75% ethanol by heating refluxing, then concentrating the extract solution, and suspending the extract in 3000 ml water, then extracting by 3000 ml water and 3000 ml n-butanol for twice. The concentrated n-butanol extract then be subjected to silica gel column chromatography (45˜75 um), and stepwise eluted by CH₃Cl/CH₃OH/H₂O solution (8:2.0:0.1) and methanol. The eluted solution be vaporized in vacuum and incorporate the crystals of component fractions of 8˜17, and subjected to ODS column chromatography, then stepwise eluted by Methanol/H₂O solutions (1:1; 65:35; 80:20). The fraction eluted with 65% methanol was prepared by Rp-18 HPLC (70% methanol), and the chromatography peak at 40 min (Rt) was collected, then drying the collection under reduced pressure to get PPD compound (100 mg).

Phoysicochemical Parameters of Steroidal Saponin Compounds MPD and PPD Synthesized in Example 1 and 2:

Methyl Protodioscin (MPD):

White powder; mp 230-233° C. (dec), [α]²⁵_{D}−88.7° (c:0.80 pyridine);

Shows positive reaction to Liebermann-Burchard, Molish and Ehrlich;

Glucose and rhamnose were detected by acid hydrolysis.
IRₘₐₓ: 3400-3450 (OH), 2950, 1380, 1040 (glycosyl C—O);
FAB-MS: 1085 (M+Na)⁺, 1062 (M+H)^{+,} 1031 (M+H—CH₃OH)⁺, 869 (M×H—CH₃OH-Glc)⁺, 723 (M+H—CH₃OH-Glc-Rha)⁺, 577 (M+H—CH₃OH-Glc-Rha×2)⁺, 415 (M+H—CH₃OH-Glc×2-Rha×2)⁺, 397 (M+H—CH₃OH—H₂O-Glc×2-Rha×2)⁺;
¹H-NMR(C₅D₅N) δ:0.87 (3H, s,CH₃-18), 0.98 (3H, d, CH₃-27), 1.08(3H, s, CH₃-19), 1.03 (3H, d, CH₃-21), 1.26 (3H, d, J=6.2 Hz), 1.28(3H,d,J=6.2 Hz).
¹³C-NMR: data please see Table 2.

Pseudoprotodioscin (PPD):

White powder, mp 174-176° C. (dec), [α]25 D−64.1° (c:0.003pyridine);

Showed positive reaction to Liebermann-Burchard, Molish and Ehrlich;

Glucose and rhamnose were detected by acid hydrolysis;

IRₘₐₓ: 3420 (OH), 2940 (CH), 1645, 1450, 1375, 1335, 1225, 1115, 1070, 1045, 920, 890. ESI-MS: 1053 (M+Na)⁺, 1029 (M−H)⁻, 883 (M−H⁻146)⁻, 737 (M−H-146×2)⁻;
¹H-NMR(C₅D₅N) δ:0.72(3H, s,CH₃-18), 1.01 (3H, d, J=6.6 Hz, CH₃-27), 1.05(3H,s,CH₃-19), 1.63(3H,s, CH₃-21), 1.62 (3H, d, J=6.0 Hz), 1.76 (3H, d, J=6.3 Hz), 4.83 (1H, d, J=7.5 Hz), 4.94 (1H, d, J=6.6 Hz), 5.32 (1H, brs, H-6), 5.85 (1H, s), 6.39 (1H, s);

¹³C-NMR: data please see Table 1:

TABLE 1
¹³C-NMR data of Pseudoprotodioscin (PPD) (C₅D₅N)

| No. | Aglycone moiety | No. | Sugar moiety |
|---|---|---|---|
| 1 | 38.0 | Glc(inner) | |
| 2 | 30.7 | 1 | 100.8 |
| 3 | 78.5 | 2 | 79.0 |
| 4 | 39.5 | 3 | 77.5 |
| 5 | 141.3 | 4 | 79.1 |
| 6 | 122.3 | 5 | 78.3 |
| 7 | 32.9 | 6 | 62.8 |
| 8 | 32.0 | Rha(1-2) | |
| 9 | 50.8 | 1 | 102.5 |
| 10 | 37.6 | 2 | 73.0 |
| 11 | 21.8 | 3 | 73.3 |
| 12 | 40.1 | 4 | 74.6 |
| 13 | 43.9 | 5 | 70.0 |
| 14 | 55.4 | 6 | 19.1 |
| 15 | 35.0 | Rha(1-4) | |
| 16 | 85.0 | 1 | 103.4 |
| 17 | 65.0 | 2 | 73.0 |
| 18 | 14.6 | 3 | 73.3 |
| 19 | 19.9 | 4 | 74.4 |
| 20 | 104.1 | 5 | 70.9 |
| 21 | 12.3 | 6 | 19.0 |
| 22 | 152.9 | Glc(−26) | |
| 23 | 34.0 | 1 | 105.4 |
| 24 | 24.2 | 2 | 75.7 |
| 25 | 32.0 | 3 | 79.1 |
| 26 | 75.5 | 4 | 72.2 |
| 27 | 17.9 | 5 | 78.5 |
| | 6 | 63.4 | |

^{a}Recorded on a Bruker-300 (75 MHz for ¹³C-NMR spectrometer).

TABLE 2
¹³C-NMR data of MPD (C₅D₅N)

| Position | Aglycone moiety | Position | Sugar moiety |
|---|---|---|---|
| 1 | 37.2 | Glc(inner) | |
| 2 | 30.2 | 1 | 100.3 |
| 3 | 78.2 | 2 | 78.0 |
| 4 | 39.0 | 3 | 78.2 |
| 5 | 140.9 | 4 | 78.7 |
| 6 | 121.9 | 5 | 77.0 |
| 7 | 32.2 | 6 | 61.4 |
| 8 | 31.7 | Rha(1→2) | |
| 9 | 50.4 | 1 | 102.1 |
| 10 | 37.6 | 2 | 72.6 |
| 11 | 21.1 | 3 | 72.8 |
| 12 | 40.5 | 4 | 74.2 |
| 13 | 40.8 | 5 | 69.6 |
| 14 | 56.6 | 6 | 18.6 |
| 15 | 32.4 | Rha(1→4) | |
| 16 | 81.4 | 1 | 103.0 |
| 17 | 64.2 | 2 | 72.6 |
| 18 | 16.3 | 3 | 72.9 |
| 19 | 19.5 | 4 | 74.0 |
| 20 | 40.8 | 5 | 70.5 |
| 21 | 16.3 | 6 | 18.7 |
| 22 | 112.7 | 26-O-Glc | |
| 23 | 30.9 | 1 | 105.0 |
| 24 | 28.2 | 2 | 75.3 |
| 25 | 34.3 | 3 | 78.6 |
| 26 | 75.3 | 4 | 71.8 |
| 27 | 17.2 | 5 | 78.7 |
| 22-O—CH₃ | 47.3 | 6 | 63.0 |

^{a}Recorded on a Bruker-500 (125 MHz for ¹³C-NMR spectrometer).

### EXAMPLE 3

### Influence of Steroidal Saponin Glycoside Compound MPD on Acute Miocardial Infarction to Rats and Canines

Object: To discuss the curative effect and mechanism of the MPD injection on acute miocardial infarction.

Methods: Applying the model of acute miocardial infarction caused by ligation of coronary artery, detecting miocardial infarction scope, coronary arterial flow and myocardial consumption of oxygen to observe the curative effect of MPD injection.

Results: MPD injection can reduce miocardial infarction scope of rats and canines and can improve the function of heart of them.

Conclusion: MPD injection has certain curative effect on acute miocardial infarction to rats and canines.

Key words: MPD; Miocardial Infarction

MPD belongs to saponin glycoside compound. The curative effect and mechanism of MPD on experimental miocardial infarction were observed in this experiment.

### 1. MATERIALS

### 1.1 ANIMALS IN THE EXPERIMENT

Wistar rats: male, body weight (200±20 g), provided by Beijing Tongli Laboratorial Animals Culturist.

Adult hybrid canines: six individuals, body weight (15.05±0.80 kg), female or male, provided by Beijing Tongli Laboratorial Animals Culturist.

### 1.2 DRUGS AND REAGENTS

MPD: provided by Xinsheng Yao, academician of China Academy, traditional Chinese medicine and natural drugs research center of Shenzhen.

0.9% Sodium chloride injection: provided by Beijing Double Crane Pharmaceutical Product Ltd., batch No: 030208612.

Di'ao Xinxuekang: provided by Chengdu Di'ao pharmaceutical Group Ltd., batch No: 0208096.

Diltiazem Hydrochloride Tablets (Herbesser): provided by Tianjin Tianbian pharmaceutical product Ltd., batch No: 0003003.

Nitro-group tetrazolium blue (N-BT): obtained from medical supply station of Academy of Military Medical Sciences, Batch No: 971120.

### 1.3 EXPERIMENTAL EQUIPMENT

Polygraph physiology recording (RM-6000 series, Japan photoelectricity);

Electric-respirator (SC-3 series, Shanghai);

Electromagnetic flowmeter (MF-1100 series, Japan photoelectricity);

Pressure capsule (MPU-0.5A);

Carrier wave amplifier (AP-601G);

Differentiator (ED-601G);

Blood-oxygen meter (AVL912 series, Switzerland);

Colorful multimedia patho-image analyzing system (MPIAS-500 series).

### 2. METHODS

### 2.1 PREPARING MODELS FOR MIOCARDIAL INFARCTION EXPERIMENT

Rat was anaesthetized by 3.5% chloral hydrate (10 mL/kg, by weight), then linking to electric-respirator, scraping off the fur of chest, opening thoracic cavity, exposing cardiac pericardium and then ligating the root of the left anterior descending of coronary artery (LADCA).

Canine was anaesthetized by 3% Pentobarbital Sodium (1 mL/kg), opening the chest, exposing the heart and making up a arcula cordis bed; leading on an epicardial electrode and then ligating the root of the left anterior descending of coronary artery (LADCA). Venous cannula on thigh was administered to inject drug, arterial cannula on cervical and pipe from external jugular vein to vena coronaria sinus were administered, thus the blood was obtained separately and AtV oxygen content were measured.

### 2.2 GROUPING

2.2.1 Preliminary experiment: twenty rats were randomly divided into model control group (injecting physiological saline 3 mL/kg by vena caudalis) and MPD treated group (40 mg/kg, i.v), ten rats per group.

2.2.2 Repeated experiment: fifty rats were randomly divided into model control group (injecting physiological saline 3 ml/kg by vena caudalis), Di'ao Xinxuekang group (administering 40 mg/kg by intragastric administration), MPD dose-intensive group (80 mg/kg by vena caudalis injection), MPD moderate dose group (40 mg/kg) and MPD low-dose group (20 mg/kg), ten rats per group. Rats were treated after 30 minutes of preparing model successfully, and were executed after 24 hours, and observing the results.

2.2.3 Six canines were randomly divided into model control group (injecting physiological saline 1 mL/1 kg by femoral vein), positive control group (Diltiazem Hydrochloride solution 0.5 mg/kg) and MPD treated group (20 mg/kg by femoral vein), two canines per group. The data of N-ST, Σ-ST, the blood oxygen content of vena coronaria sinus and artery, and the scope of miocardial infarction of pro-administration were collected after administration at once, and 5, 15, 30, 60, 120, 180 minutes after administration.

### 2.3 OBSERVING PARAMETERS

Epicardial electrogram: recording the change of N-ST and Σ-ST.

Determining the scope of miocardial infarction (N-BT staining method): quickly taking out the heart from the executed animal, then washing by physiologic saline and dewatering with filter paper, the heart was cut into 4 pieces uniformly from apex of heart to ligating thread, then the pieces were put into N-BT staining solution, keeping in common temperature, avoiding light in 2 minutes. Then measuring the size of each slice, and the size of miocardial infarction (non-staining zone with N-BT) by colorful multimedia patho-image analytical system, total area of ventricular muscle, total area of infarction of ventricular muscle, and the ratio of miocardial infarction size relative to the size of ventricles were measured respectively.

### 2.4 STATISTICAL ANALYSIS

Applying SPSS10.0 in statistical analysis, the data were represented by means of X̅±SD.

### 3. RESULTS OF THE EXPERIMENT

### 3.1 EFFECT SCOPE OF MPD ON THE MIOCARDIAL INFARCTION TO RAT IN PRELIMINARY EXPERIMENT

As showing in table 3 & FIG. 1, miocardial infarction size compare to the size of ventricles in model control group is 41.20±12.25 (%), this result means modeling was successful. Miocardial infarction size compare to the size of ventricles in MPD treated group is 33.4±8.09 (%), it is significant different compare to model control group.

TABLE 3
Effect scope of MPD on the miocardial infarction to rat
in preliminary experiment.( X̅ ± SD)

| | | dosage | miocardial infarction size/ |
|---|---|---|---|
| Groups | n | (/kg) | ventricular size (%) |
| Model control group | 10 | 3 ml | 41.20 ± 12.25 |
| MPD group | 10 | 40 mg | 33.40 ± 8.09** |

Note:
comparing with model control group
**P < 0.01

### 3.2 EFFECT SCOPE OF MPD ON THE MIOCARDIAL INFARCTION TO RAT IN REPEATING EXPERIMENT

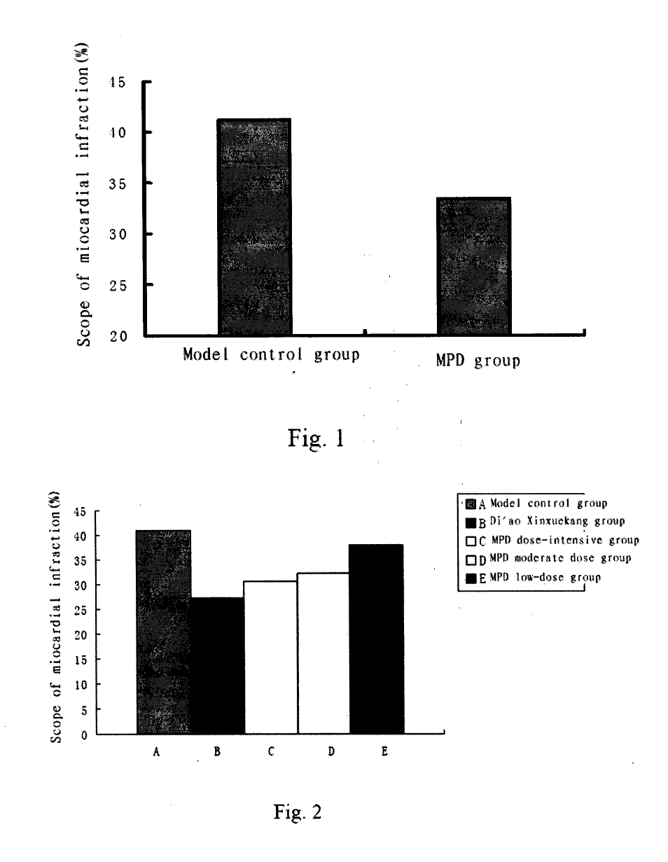

As showing in table 4 & FIG. 2, miocardial infarction size compare to the size of ventricles in model control group is 40.99±6.64 (%), this result means modeling was successful. Miocardial infarction size compare to the size of ventricles in Di'ao Xinxuekang group is 27.24±10.24 (%). The scope of miocardial infarction in MPD group is more narrower, comparing with model control group, MPD dose-intensive group (30.62±9.46%) has extremely significant difference, MPD moderate dose group (32.32±6.92%) has significant difference, MPD low-dose group (37.89±8.41%) has diminished tendency, but has no significant statistical difference.

TABLE 4
Effect scope of MPD on the miocardial infarction to rat
in repeated experiment( X̅ ± SD)

| | | | miocardial infarction |
|---|---|---|---|
| | | dosage | size/ventricular |
| Groups | n | (/kg) | size (%) |
| Model control group | 10 | 3 ml | 40.99 ± 6.64 |
| Di'ao Xinxuekang group | 10 | 40 mg | 27.24 ± 10.24** |
| MPD dose-intensive group | 10 | 80 mg | 30.62 ± 9.46** |
| MPD moderate dose group | 10 | 40 mg | 32.32 ± 6.92* |
| MPD low-dose group | 10 | 20 mg | 37.89 ± 8.41 |

Note:
Comparing with model control group
*P < 0.05,
**P < 0.01

### 3.3 EFFECT SCOPE OF MPD ON THE MIOCARDIAL INFARCTION TO CANINES

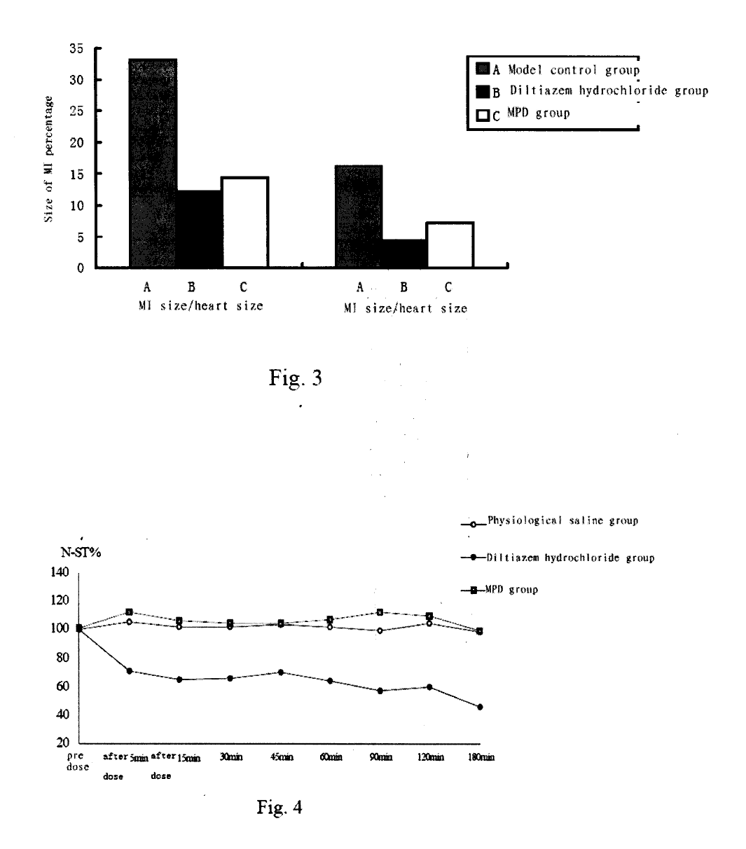

As showing in table 5 & FIG. 3, miocardial infarction size compare to the size of heart in model control group is 6.45±1.03 (%), and compare to the size of ventricles is 16.21±1.00 (%). Miocardial infarction size compare to the size of heart in MPD group is 2.74±0.33 (%), and compare to the size of ventricles is 7.30±0.97 (%), these two groups are significant different. Diltiazem Hydrochloride group has significant difference comparing with model control group also

TABLE 5
Effect scope of MPD on the miocardial infarction to canines. ( X̅ ± SD)

| | | | | Infarction size/ |
|---|---|---|---|---|
| | | dosage | Infarction size/ | ventricles size |
| Groups | n | (/kg) | heart size (%) | (%) |
| Model contrast group | 2 | 1 ml | 6.45 ± 1.03 | 16.21 ± 1.00 |
| Diltiazem | 2 | 0.5 mg | 1.81 ± 0.79** | 4.36 ± 1.15** |
| Hydrochloride | | | | |
| group | | | | |
| MPD group | 2 | 20 mg | 2.74 ± 0.33** | 7.30 ± 0.97** |

Note:
Comparing with model control group
**P < 0.01

### 3.4 INFLUENCE OF MPD ON EPICARDIAL ELECTROGRAM TO CANINES

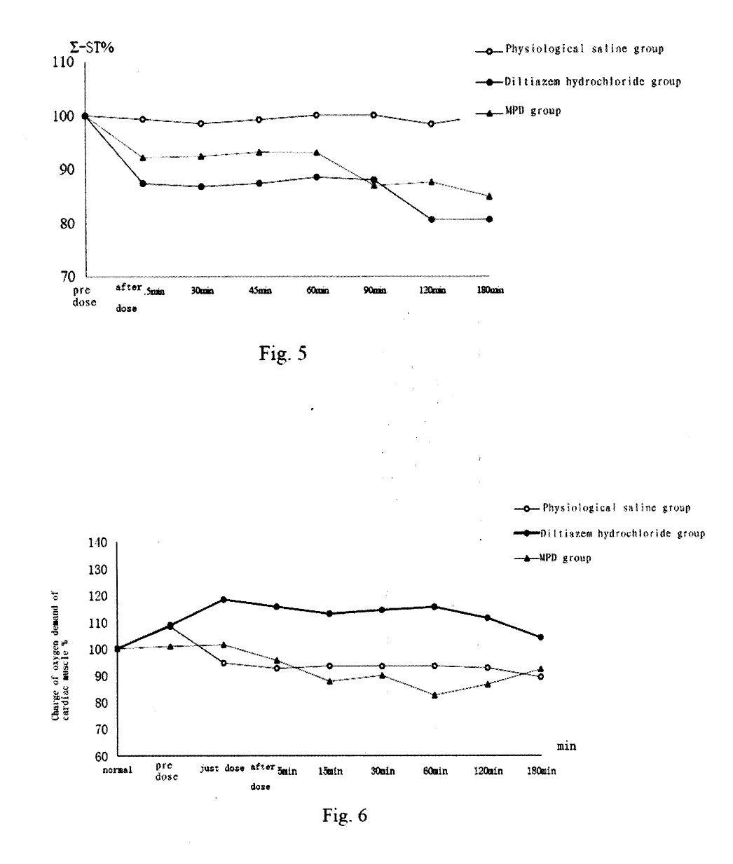

As shown in FIGS. 4 and 5, MPD treated group has no significant difference in N-ST comparing with control group, but both of the treating groups have significant decrease in Σ-ST comparing with control group.

### 3.5 INFLUENCE OF MPD ON CORONARY ARTERIAL FLOW AND MIOCARDIAL CONSUMPTION OF OXYGEN (MCO) TO CANINES

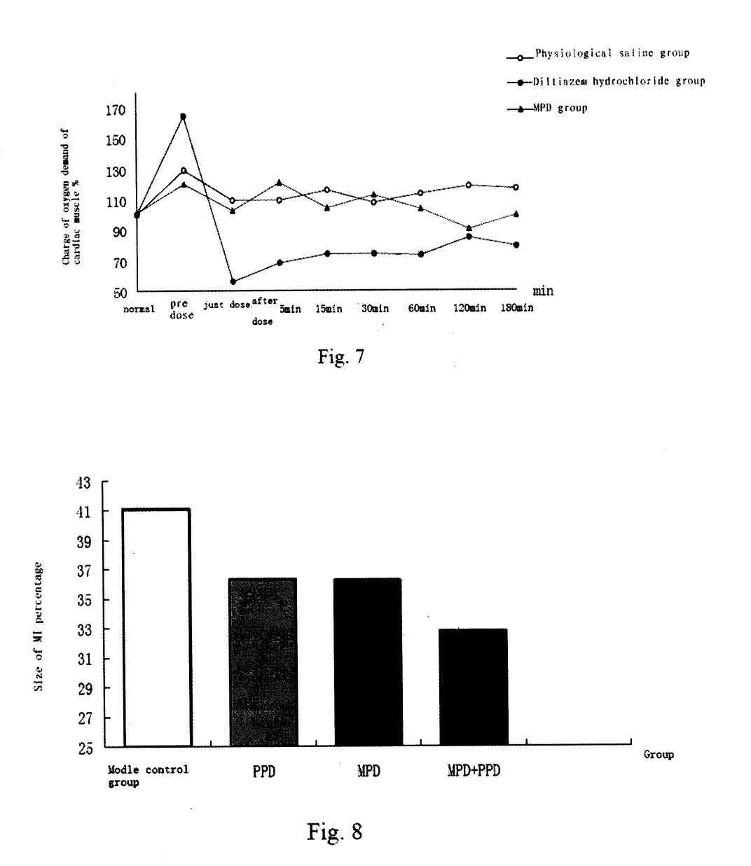

As shown in FIGS. 6 and 7, MPD treated group had no significant difference in the flow of aeteria coronaria and miocardial consumption of oxygen (MCO) comparing with control group.

### 4. DISCUSSING

Di'ao Xinxuekang has the efficiency of enhancing the dilatation of coronary artery blood vessel, and improving the effect of blood losing of cardiac muscle, it usually using for curing coronary artery disease, so it be used as positive control drug in this study. MPD belongs to saponin glycoside compound. The results of the two experiments indicated that MPD has the effect of improving miocardial infarction which caused by coronary artery ligation to rats. Comparing with model group, the infarction size of MPD dose-intensive group is extremely significantly diminished, the moderate dose group is significant difference, and the low-dose group has descending tendency. The experiments also indicated that Di'ao Xinxuekang has the effect of improving miocardial infarction to rats, and there's no significance difference between Di'ao Xinxuekang and MPD. Miocardial infarction experiment to canines also indicated that obvious efficiency has achieved in curing miocardial infarction by vein administering MPD.

### EXAMPLE 4

### Influence of Steroidal Saponin Glycoside Compound PPD on Acute Myocardial Infarction to Rats

Object: To study therapeutical effect of PPD on acute miocardial infarction (AMI).

Methods: Rat model of AMI was established by ligating of coronary artery. Miocardial infarction scope to rats was observed to ascertain the effect of PPD.

Results: PPD and MPD have obvious effect scope on miocardial infarction.

Conclusion: MPD and PPD can reduce miocardial infarction scope to rats (P<0.05), and MPD is a little better than PPD.

### 1. MATERIALS

### 1.1 ANIMALS IN THE EXPERIMENT

Wistar rats: male, body weight (170±20 g), provided by Beijing Tongli Laboratorial Animals Culturist.

### 1.2 DRUGS AND REAGENTS

MPD, PPD: provided by Xinsheng Yao, academician of China Academy, traditional Chinese medicine and natural drugs research center of Shenzhen.

0.9% sodium chloride injection: provided by Beijing Double Crane Pharmaceutical Product Ltd., batch No: 030208612.

Nitro-group tetrazolium blue (N-BT): obtained from medical supply station of Academy of Military Medical Sciences, Batch No: 971120.

### 1.3 EXPERIMENTAL EQUIPMENT

Electric-respirator (SC-3 series, Shanghai);

Colorful multimedia patho-image analyzing system (MPIAS-500 series).

### 2. METHODS

### 2.1 PREPARING MODELS FOR MIOCARDIAL INFARCTION EXPERIMENT

Rat was anaesthetized by 3.5% chloral hydrate (10 mL/kg, by weight), then linking to electric-respirator, scraping off the fur of chest, opening thoracic cavity, exposing cardiac pericardium and then ligating the root of the left anterior descending of coronary artery (LADCA).

### 2.2 GROUPING

### 2.2.1 EXPERIMENT 1

12 rats were randomly divided into model control group (physiological saline 5 mL/kg by pouring down throat) and PPD treated group (40 mg/5 mL/kg, by pouring to stomach), 6 rats per group. Rats were administrated one time after ligating, then be executed 24 hours later.

### 2.2.2 EXPERIMENT 2 (REPEATED EXPERIMENT)

24 rats were randomly divided into model control group (physiological saline 5 mL/kg by pouring to stomach), MPD treated group (40 mg/5 mL/kg, by pouring to stomach) and PPD treated group (40 mg/5 mL/kg, by pouring to stomach). Rats were administrated one time after ligating, then be executed 24 hours later.

### 2.3 OBSERVING PARAMETERS

Determining the scope of miocardial infarction (N-BT staining method): quickly taking out the heart from the executed animal, then washing by physiologic saline and dewatering with filter paper, the heart was cut into 5 pieces uniformly from apex of heart to ligating thread, then the pieces were put into N-BT staining solution, keeping in common temperature, avoiding light in 2 minutes. Then measuring the size of each slice, and the size of miocardial infarction (non-staining zone with N-BT) by colorful multimedia patho-image analytical system, total area of ventricular muscle, total area of infarction of ventricular muscle, and the ratio of miocardial infarction size relative to the size of ventricles were measured respectively.

### 2.4 STATISTICAL TREATMENT

Applying SPSS10.0 in statistical analysis, the data were represented by means of X̅±SD.

### 3. RESULTS

### 3.1 EXPERIMENT 1: EFFECT SCOPE OF PPD ON THE MIOCARDIAL INFARCTION TO RAT

As showing in table 6, miocardial infarction size compare to the size of ventricles in model control group is 42.48±3.88 (%), this result means modeling was successful. Miocardial infarction size compare to the size of ventricles in PPD treated group is 36.25±7.20 (%), it is significant different compare to model control group (P<0.05).

TABLE 6
Effect scope of MPD on the miocardial infarction to rat ( X̅ ± SD)

| | | dosage | Miocardial infarction size/ |
|---|---|---|---|
| groups | n | (/kg) | ventricular size (%) |
| Model control group | 6 | 5 ml | 42.48 ± 3.88 |
| PPD group | 6 | 40 mg | 36.25 ± 7.20* |

Note:
Comparing with model control group
*P < 0.05

### 3.2 EXPERIMENT 2: EFFECT SCOPE OF MPD & PPD ON THE MIOCARDIAL INFARCTION TO RAT (REPEATING EXPERIMENT)

As showing in table 7, the repeating experiment indicated that MPD and PPD could obviously reduce the scope of miocardial infarction, and MPD has more obvious effect on decreasing the scope of miocardial infarction, and the death rate in operation is much lower.

TABLE 7
Effect scope of MPD & PPD on the miocardial infarction to rat
( X̅ ± SD)

| | | Dosage | Infarction size/whole | Death rate in |
|---|---|---|---|---|
| Groups | n | (/kg) | heart size (%) | operation |
| Model control group | 9 | 5 ml | 41.06 ± 4.98 | 55.6% (5/9) |
| MPD group | 8 | 40 mg | 33.71 ± 6.73** | 0% (0/8) |
| PPD group | 7 | 40 mg | 36.31 ± 1.90* | 14.3% (1/7) |

Note:
Comparing with model control group
*P < 0.05,
**P < 0.01

### 4. CONCLUSION

The result of experiment 1 indicating that PPD has improving effect on miocardial infarction to rats, there's significant difference comparing with model control group. In order to validate the experimental result, experiment 2 was carried out and added MPD group, the results indicating that both MPD and PPD can reduce the scope of miocardial infarction by the administrative way of pouring to stomach, and both have significant difference comparing with model control group; MPD is a little better than PPD.

### EXAMPLE 5

### Influence of the Mixture of MPD and PPD in Various Ratio on the Acute Miocardial Infarction to Rats

Object: To study synergetic therapeutical effect of MPD and PPD mixture on acute miocardial infarction (AMI).

Methods: Rat model of AMI was established by ligating of coronary artery. Miocardial infarction scope to rats was observed to ascertain the effect of the mixture of MPD and PPD with certain proportion.

Results: MPD and PPD team up together has better efficiency than separately using one of them on myocardial infarction.

Conclusion: MPD together with PPD have synergistic effect.

### 1. MATERIALS

### 1.1 ANIMALS IN THE EXPERIMENT

Wistar rats: male, body weight (170±20 g), provided by Beijing Tongli Laboratorial Animals Culturist.

### 1.2 DRUGS AND REAGENTS

MPD, PPD: provided by Xinsheng Yao, academician of China Academy, traditional Chinese medicine and natural drugs research center of Shenzhen, and the ratio of MPD and PPD is 1:1.

0.9% sodium chloride injection: provided by Beijing Double Crane Pharmaceutical Product Ltd., batch No: 030208612.

Nitro-group tetrazolium blue (N-BT): obtained from medical supply station of Academy of Military Medical Sciences, Batch No: 971120.

### 1.3 EXPERIMENTAL EQUIPMENT

Electric-respirator (SC-3 series, Shanghai);

Colorful multimedia patho-image analyzing system (MPIAS-500 series).

### 2. METHODS

### 2.1 PREPARING MODELS FOR MIOCARDIAL INFARCTION EXPERIMENT

Rat was anaesthetized by 3.5% chloral hydrate (10 mL/kg, by weight), then linking to electric-respirator, scraping off the fur of chest, opening thoracic cavity, exposing cardiac pericardium and then ligating the root of the left anterior descending of coronary artery (LADCA).

### 2.2 GROUPING

33 rats were randomly divided into model control group (physiological saline 5 mL/kg by pouring down throat), MPD treated group (40 mg/5 mL/kg, by pouring to stomach), PPD treated group (40 mg/5 mL/kg, by pouring to stomach) and MPD+PPD treated group (40 mg/5 mL/kg, by pouring to stomach). Rats were administrated one time after ligating, then be executed 24 hours later.

### 2.3 OBSERVING PARAMETERS

Determining the scope of miocardial infarction (N-BT staining method): quickly taking out the heart from the executed animal, then washing by physiologic saline and dewatering with filter paper, the heart was cut into 5 pieces uniformly from apex of heart to ligating thread, then the pieces were put into N-BT staining solution, keeping in common temperature, avoiding light in 2 minutes. Then measuring the size of each slice, and the size of miocardial infarction (non-staining zone with N-BT) by colorful multimedia patho-image analytical system, total area of ventricular muscle, total area of infarction of ventricular muscle, and the ratio of miocardial infarction size relative to the size of ventricles were measured respectively.

### 2.4 STATISTICAL TREATMENT

Applying SPSS10.0 in statistical analysis, the data were represented by means of X̅±SD.

### 3. RESULTS

As showing in table 8 & FIG. 8, miocardial infarction size compare to the size of ventricles in model control group is 41.06±1.66 (%), this result means modeling was successful. Miocardial infarction size compare to the size of ventricles in MPD treated group is 36.24±3.74 (%). miocardial infarction size compare to the size of ventricles in PPD treated group is 36.31±1.90 (%). Miocardial infarction size compare to the size of ventricles in MPD+PPD treated group is 32.74±4.90 (%). There has significant difference comparing with model control group (P<0.05), however, MPD+PPD treated group is the best one.

TABLE 8
Effect scope of MPD, PPD and MPD + PPD
on the miocardial infarction to rat (X ± S)

| | | dosage | infarct size |
|---|---|---|---|
| Groups | n | (/kg) | (IS) (%) |
| Model control group | 9 | 5 ml | 41.06 ± 4.98 |
| MPD group | 8 | 40 mg | 36.24 ± 3.74* |
| PPD group | 7 | 40 mg | 36.31 ± 1.90* |
| MPD + PPD | 9 | 40 mg | 32.74 ± 4.90* |

Note:
Comparing with model control group.
*P < 0.05

### 4. CONCLUSION

Both separately apply MPD, PPD and the combining of them have obvious curing effect on acute miocardial infarction to rats. The Combining of MPD and PPD by certain proportion can make synergistic effect, and has better therapeutical effect with same dosage.

1. A steroidal saponins compound with the chemical structure of: 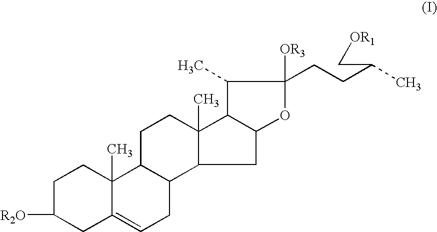
Wherein, R₁=β-D-glucose;
R₂=straight or bifurcate sugar chains including β-D-glucose, α-D-glucose, α-L-rhamnose, β-D-galactose, α-D-galactose, β-D-mannose, α-D-mannose, β-D-arabinose, α-D-arabinose, β-D-xylose, α-D-xylose, β-D-ribose, α-D-ribose, β-D-lyxose, α-D-lyxose, α-D-fucose, and 6-deoxysugars, and 2,6-dideozysugars corresponding to each of foresaid aldohexoses;
R₃═H or CH₃.

2. A steroidal saponins compound of claim 1, wherein the chemical structure is:

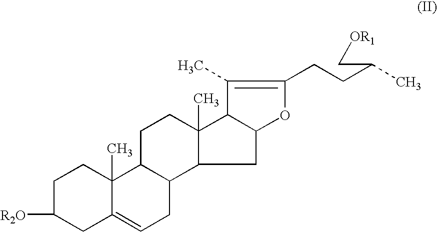

3. The steroidal saponins of claim 1, wherein in the chemical structure (I):

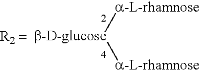

4. The steroidal saponins of claim 2, wherein in the chemical structure (II):

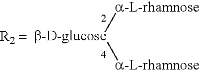

5-7. (canceled)

8. The steroidal saponins of claim 1, wherein Phoysicochemical parameters are:
White powder; mp 230-233° C. (dec), [α]²⁵_{D}−88.7° (c:0.80 pyridine);
Shows positive reaction to Liebermann-Burchard, Molish and Ehrlich;
Glucose and rhamnose were detected by acid hydrolysis.
IRₘₐₓ: 3400-3450 (OH), 2950, 1380, 1040 (glycosyl C—O);
FAB-MS: 1085 (M+Na)⁺, 1062 (M+H)^{+,} 1031 (M+H—CH₃OH)⁺, 869 (M×H—CH₃OH-Glc)⁻, 723 (M+H—CH₃OH-Glc-Rha)⁺, 577 (M+H—CH₃OH-Glc-Rha×2)⁺, 415 (M+H—CH₃OH-Glc×2-Rha×2)⁺, 397 (M+H—CH₃OH—H₂O-Glc×2-Rha×2)⁺;
¹H-NMR(C₅D₅N) δ:0.87 (3H, s,CH₃-18), 0.98 (3H, d, CH₃-27), 1.08(3H, s, CH₃-19), 1.03 (3H, d, CH₃-21), 1.26 (3H, d, J=6.2 Hz), 1.28(3H,d,J=6.2 Hz).
¹³C-NMR: data please see Table 2.

9. The steroidal saponins of claim 2, wherein Phoysicochemical parameters are:
White powder, mp 174-176° C. (dec), [α]25 D−64.1° (c:0.003 pyridine);
Showed positive reaction to Liebermann-Burchard, Molish and Ehrlich;
Glucose and rhamnose were detected by acid hydrolysis;
IRₘₐₓ: 3420 (OH), 2940 (CH), 1645, 1450, 1375, 1335, 1225, 1115, 1070, 1045, 920, 890. ESI-MS: 1053 (M+Na)⁺, 1029 (M−H)⁻, 883 (M−H-146)⁻, 737 (M−H-146×2)⁻;
¹H-NMR(C₅D₅N) δ:0.72(3H, s,CH₃-18), 1.01 (3H, d, J=6.6 Hz, CH₃-27), 1.05(3H,s,CH₃-19), 1.63(3H,s, CH₃-21), 1.62 (3H, d, J=6.0 Hz), 1.76 (3H, d, J=6.3 Hz), 4.83 (1H, d, J=7.5 Hz), 4.94 (1H, d, J=6.6 Hz), 5.32 (1H, brs, H-6), 5.85 (1H, s), 6.39 (1H, s);
¹³C-NMR: data please see Table 1:

10. A method for producing the steroidal saponins compound of claim 1, wherein comprising the steps of:
1) extracting fresh rhizome of Discorea nipponica with 80% ethanol by heating refluxing; then concentrating the extracted liquid and suspending the extract in water to get dissolved portion and unsolved portion;
2) passing the dissolved portion through D101 absorbent resin column, and eluting by distilled water first, then by 10%, 50% and 95% ethanol in order;
3) concentrating the 50% ethanol eluted solution, and subjecting to silica gel column chromatography with granularity of 45˜75 um, then eluting by CH₃Cl, CH₃OH and H₂O mixture solution in ratio of 8:2.5:0.01, and methanol step by step; vaporizing and concentrating the eluted solution under decreased pressure, and incorporating the crystals of component fractions of 46˜50, then re-crystallizing the crystals to get MPD compound.

11. A method for producing the steroidal saponins compound of claim 2, wherein comprising the steps of:
1) extracting rhizome of Discorea futschauensis with 75% ethanol by heating refluxing, then concentrating the extract solution, and suspending the extract in 3000 ml water;
2) extracting the suspending solution by 3000 ml water and 3000 ml n-butanol for twice, and subjecting the concentrated n-butanol extract to silica gel column chromatography with granularity of 45˜75 um; then eluting by CH₃Cl, CH₃OH and H₂O mixture solution in ratio of 8:2.0:0.1 and methanol step by step;
3) vaporizing and concentrating the eluted solution under decreased pressure, and incorporate the distillate of 8˜17, and subjecting to ODS column chromatography; then eluting by methanol and H₂O mixture solutions in ratio of 1:1, 65:35 and 80:20 step by step, collecting the fraction eluted by 65% methanol and preparing by Rp-18 HPLC with 70% methanol, and then collecting the chromatography peak at 40 min; drying the collection under reduced pressure to get PPD compound.

12. The application of steroidal saponins compound for curing the diseases miocardial infarction, coronary artery disease, heart angina, arrhythmia, blood losing of cardiac muscle, hypertension, hyperlipaemia and ropy blood.

13. The application of steroidal saponins compound of claim 12, wherein said steroidal saponins is MethylProtodioscin (MPD).

14. The application of steroidal saponins compounds of claim 12, wherein said steroidal saponins compounds is Pseudoprotodioscin (PPD).

15. The application of steroidal saponins compounds of claim 12, wherein said steroidal saponins compounds is a composition of MethylProtodioscin (MPD) and Pseudoprotodioscin (PPD).
